(19) United States
(12) Patent Application Publication
(10) Pub. No.: US 2008/0188794 A1
(43) Pub. Date: Aug. 7, 2008

(54) PERCUTANEOUS TENTING

(75) Inventors: Mark S. Oritz, Milford, OH (US); Ragae M. Ghabrial, Cincinnati, OH (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: Ethicon Endo-Surgery, Inc., Cincinnati, OH (US)

(21) Appl. No.: 11/670,487

(22) Filed: Feb. 2, 2007

(51) Int. Cl. A61M 31/00

(52) U.S. Cl. 604/48

(57) ABSTRACT

Methods and devices are provided for manipulating tissue and creating a space in a body cavity. In an exemplary embodiment, a space-creating frame can be delivered and deployed within a body cavity, a cable can be manipulated to tent a tissue wall of the body cavity, and an illumination source and an imaging apparatus can be activated to provide visibility within the body cavity.

### FIELD OF THE INVENTION

The present invention relates to methods and devices for performing surgical procedures, and in particular to methods and devices for manipulating tissue and creating a space in a body cavity.

### BACKGROUND OF THE INVENTION

In certain minimally invasive surgical procedures, e.g., endoscopic and laparoscopic surgeries, a surgeon performs diagnostic and therapeutic procedures at the surgical site through a natural body aperture or through one or more small incisions, using instruments specially designed for this purpose. Problems encountered by a surgeon in such minimally invasive surgical procedures include reduced visibility and field of vision. Other problems that emerge during such surgeries include limited availability of space for the use of additional tools, since existing access devices, such as endoscopes, take up nearly all of the afforded space.

Further, some minimally invasive surgical procedures employ insufflation techniques to create a working and viewing space in the target lumen or organ. However, insufflation can lead to several problems. For example, the pressure required to insufflate the abdominal cavity can cause the stomach to collapse. Difficulties with insufflating only the target body cavity can also arise.

Accordingly, there is a need for methods and devices for manipulating tissue and creating space in a body cavity, which avoid the need for insufflation techniques, and provided enhanced visibility and field of vision.

### SUMMARY OF THE INVENTION

The present invention generally provides methods and devices for manipulating tissue and creating space in a body cavity, particularly during minimally invasive surgical procedures. In one embodiment, a device for manipulating tissue can include a first elongate member with a cable coupled thereto, a second elongate member slidably disposed on the cable and movable between a first position in which the first elongate member and second elongate member are configured to be inserted through a hollow tube, and a second position in which the first elongate member and second elongate member mate to one another and are configured to engage tissue. The device can further include an imaging apparatus disposed on at least one of the first elongate member, the second elongate member, and the cable. In one aspect, the imaging apparatus is at least one of a CCD chip and a CMOS chip.

The cable of the device of the present invention can be manipulated to move the tissue. The cable can also include a proximal end having a piercing tip. In one aspect, the piercing tip can be in the form of a needle.

The first and second elongate members can be mated to another in a variety of ways. In one embodiment, a first surface of the first elongate member has a first mating structure formed therein and a mating surface of the second elongate member has a second mating structure formed thereon, the second mating structure being complementary with the first mating structure. The first and second mating structures can be configured to engage each other when the first and second elongate members are operatively mated. In another aspect, the first elongate member and the second elongate member mate in a substantially perpendicular orientation.

In another embodiment, at least one of the first elongate member, the second elongate member, and the cable can further include at least one illumination element coupled thereto. In one aspect, the illumination element can be in the form of a light-emitting diode.

In yet another embodiment, the device can also include an actuation mechanism to enable operation of the imaging apparatus, and an actuation mechanism effective to enable operation of the illumination element. In one aspect, the cable communicates a signal from the imaging apparatus. In another aspect, a wire, which communicates a signal from the imaging apparatus, is adjacent the cable.

In one embodiment, an exemplary method for creating a space in a body cavity can include delivering a tissue-tenting apparatus coupled to a cable in a delivery configuration to a surgical site through a natural body orifice, manipulating the cable to configure the tissue-tenting apparatus in a deployed configuration such that at least a portion of the tenting apparatus is capable of lifting tissue at the surgical site, and activating an imaging apparatus formed on one of the tenting apparatus and the cable to enable visualization of the surgical site.

In one aspect, the surgical site is the stomach. In another aspect, the tenting apparatus, in the deployed configuration, is in the form of a first and second elongate members mated to one another in a substantially perpendicular orientation.

In another embodiment, manipulating the cable is effected by breaching the stomach wall and the peritoneal wall with a proximal portion of the cable such that it exits the body cavity, and exerting a pulling force on the cable. Following manipulation of the cable, the exemplary method can further include activating an illumination source associated with one of the tenting apparatus and the cable.

### BRIEF DESCRIPTION OF THE DRAWINGS

The invention will be more fully understood from the following detailed description taken in conjunction with the accompanying drawings, in which:

FIG. 1 is a perspective view of an embodiment of a device for manipulating tissue, showing the device in a delivery configuration;

FIG. 2 is a perspective view showing one embodiment of the device of FIG. 1 in a deployed configuration;

FIG. 3 is a perspective view illustrating one embodiment of an exemplary method for creating a space in a body cavity, showing the device of FIG. 1 in a delivery configuration and positioned within a body cavity; and

FIG. 4 is a perspective view of another embodiment of the exemplary method of FIG. 3, showing the device of FIG. 1 in a deployed configuration and lifting a wall of the body cavity.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present invention generally provides methods and devices for manipulating tissue, and in particular for tenting a wall of a body cavity to create a space within the body cavity. The term “body cavity” is used herein in its broad sense and includes any hollow lumen within the body, such as organs, as well as cavities within the body, such as the peritoneal cavity.

The methods and devices described herein can include introducing and deploying a space-creating frame within a body cavity, manipulating a lifting wire to tent a tissue wall of the body cavity, and activating an illumination source and imaging apparatus to provide visibility within the body cavity. Effecting the manipulation of tissue and creation of a space within a body cavity with a deployable apparatus is advantageous in that it avoids the complications associated with insufflation of body cavities. In addition, the use of a deployable apparatus that includes illumination and imaging components provides enhanced illumination and visibility in the space created by the deployable apparatus.

While the devices and methods disclosed herein can be used in conventional, open surgical procedures, they are particularly useful in minimally invasive surgical procedures, such as laparoscopic and endoscopic procedures. The principles described herein can be applicable to the particular types of tools described herein, and to a variety of other surgical tools having similar functions. In addition, the tools can be used alone in a surgical procedure, or they can be used in conjunction with other devices, such as endoscopes, that facilitate minimally invasive surgical procedures.

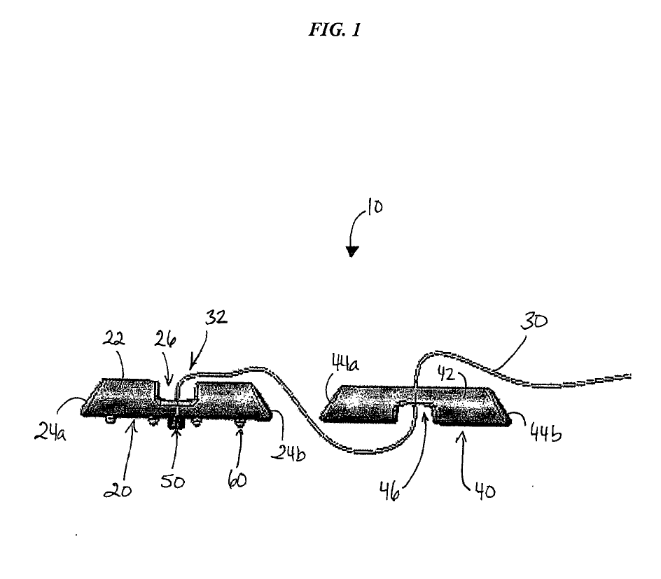

FIG. 1 illustrates one exemplary embodiment of a device 10 for manipulating tissue to create a space within a body cavity. The device 10 can generally include a first elongate member 20 with a cable 30 coupled thereto. The device 10 can further include a second elongate member 40 coupled to the cable 30. Although FIG. 1 illustrates two elongate members mated to the cable 30, the present invention also encompasses embodiments wherein only one elongate member is employed. Alternatively, multiple elongate members can disposed on and utilized with the cable 30. The device 10 can also include an imaging apparatus 50 disposed on either of the first elongate member 20, the second elongate member 40, and/or the cable 30.

The first elongate member 20 can be fixably mated to the distal end 32 of the cable 30 with the second elongate member 40 slidably disposed thereon. The second elongate member 40 can be configured such that is movable between a first position and a second position. FIG. 1 depicts the first position in which the device 10 adopts a delivery configuration such that the first elongate member 20 and the second elongate member 40 are configured to be inserted into a body cavity through, for example, a hollow tube or passageway. In another embodiment, the first elongate member 20 can also be slidably movable along the length of the cable 30. Alternatively, the second elongate member 40 can be fixably mated to the cable 30.

The first elongate member 20 and the cable 30 can be coupled to one another in a variety of ways, and one or both of the first and second elongate members 20, 40 can have mating features, as described below, to facilitate mating. In one embodiment, the cable 30 is coupled to an intermediate portion of the body 22 of the first elongate member 20. One skilled in the art will appreciate that the first elongate member 20 and the cable 30 can be coupled to one another along any portion or length thereof, such that the cable 30 and the first elongate member 20 are configured to cooperate to move tissue. Further, as shown in FIG. 1, the first elongate member 20 can be positioned at the distal end 32 of the cable 30 such that the imaging apparatus 50 is adjacent to or forms a terminal part of cable 30. In another embodiment, the first elongate member 20 can be positioned at any point along the length of the cable 30 that is suitable to enable the device 10, when deployed, to create a space. The first elongate member 20 can be mated to the cable using a variety of techniques known in the art. For example, the first elongate member 20 can be integrally formed with the distal end 32 of the cable 30, or permanently mated thereto. Alternatively, the first elongate member 20 can be removably attached to distal end 32 of the cable 30.

The first and second elongate members can have virtually any configuration that enables them to manipulate tissue and tent a wall of a body cavity. For example, the device 10 can be constructed as a deployable mechanical wall lift, or formed from a set of elongate members that can be assembled, in situ, into the form of a cross-bar. In another example, the device 10 can be formed from a large spiral screw, which may be introduced percutaneously, and positioned to accommodate atraumatic lifting of a tissue or lumen wall. Alternatively, the device 10 can be formed from a large extra-corporal vacuum cup that facilitates atraumatic lifting of tissue or a lumen wall from outside the body.

The first and second elongate members 20, 40, can further include various shapes and dimensions. As shown in FIG. 1, the body 22, 42 of the first and second elongate members 20, 40 can have a substantially circular cross-sectional shape. By way of non-limiting example, other suitable cross-sectional shapes for the elongate member bodies 22, 42 include square, oval, triangular, and trapezoidal. FIG. 1 further illustrates the first and second elongate members 20, 40 having opposing ends 24a, 24b, 44a, 44b with a slanted configuration. While such beveled opposing ends are shown, one skilled in the art will appreciate that the opposing ends 24a, 24b, 44a, 44b can have any configuration that prevents or minimizes the tearing or traumatizing of tissue when the elongate members 20, 40 come into contact with tissue. For example, the opposing ends 24a, 24b, 44a, 44b can have a rounded configuration, or a substantially square shape with rounded edges. The shape and dimensions of the device 10, particularly the elongate members 20, 40, will vary depending on the intended surgical technique and application.

As described above, the cable 30 of the device 10 can have a distal end 32 and a proximal end (not shown). The proximal end of the cable 30 can include a piercing tip, which in one embodiment, can be in the form of a needle. It will be understood by one skilled in the art that the piercing tip can have any shape or configuration that enables the proximal end of the cable 30 to breach tissue or a body cavity wall.

The cable 30 can be formed in varying lengths and thickness, but it should be suitable to enable the cable 30 to be passed transmurally such that the distal end 32 remains within a body cavity, while the proximal end extends to a location external to the body cavity. The thickness of the cable 30 can have any circumference suitable for enabling the elongate members 20, 40 to be slidably movable along the cable. Further, the cable 30 can have a strength sufficient to support the elongate members, as well as to absorb the stress associated with lifting and tenting tissue or a wall of a body cavity.

As illustrated in FIG. 1, the device 10 can further include at least one illumination element 60, which can be coupled to a surface of the first elongate member 20. The illumination element 60 can be formed from any component capable of providing a light source. In one exemplary embodiment, the illumination element 60 is a light-emitting diode. While the illumination element 60 is shown coupled to the first elongate member 20, one skilled in the art will appreciate that the illumination element 60 can be coupled to any suitable portion of the device 10. For example, the illumination element 60 can be coupled to the second elongate member 40 or the cable 30. In addition, the first and second elongate members 20, 40, and cable 30 can include more than one illumination element 60 coupled thereto. The coupling of the illumination element 60 to the various parts of the device 10 can be achieved in a variety of known mating techniques. For example, the illumination element 60 can be integrally formed with a component of the device 10, or fixedly mated therewith. Alternatively, the illumination element 60 can be removably attached to the device 10.

The present invention further provides for an imaging apparatus 50 disposed on at least one of the first elongate member 20, the second elongate member 40, and the cable 30. In one exemplary embodiment, as illustrated in FIG. 1, the imaging apparatus 50 can be coupled to a distal end 32 of the cable 30 such that it is positioned in contact with a surface of the first elongate member 20. The imaging apparatus 50 can be formed from any component that acts as an image sensor, such as a CCD chip or a CMOS chip. The imaging apparatus 50 can be attached to the device 10 with a variety of known techniques, as discussed with respect to the illumination element 60. In addition, the device 10 can include more than one imaging apparatus 50 coupled thereto.

The device 10 can also include an actuation mechanism to enable operation of at least one of the illumination element 60. An actuation mechanism to enable operation of the imaging apparatus 50 can further be included. In an exemplary embodiment, the cable 30 can serve as the actuation mechanism providing a signal wire and a power wire for operation of the illumination element 60 and the imaging apparatus 50. Alternatively, a wire communicating a signal from the imaging apparatus 50 and/or the illumination element 60 can be separately provided and positioned adjacent the cable 30.

In general, the various components of the device 10 can be formed from virtually any material suitable for use within a patient's body. By way of non-limiting example, suitable materials include metals such as aluminum, stainless steel or titanium, shape-memory materials, polymers, or various other biocompatible materials. The components of the device 10 can also be rigid or flexible. In one exemplary embodiment, the elongate members 20, 40 and the cable 30 are sufficiently flexible to allow the elongate members 20, 40 and the cable 30 to be inserted endoscopically through a natural orifice and through a tortuous body lumen. In another embodiment, the elongate members 20, 40 can be sufficiently rigid to enable manipulation of tissue when coming into contact therewith. The cable 30 can also have sufficient rigidity to effectively transmit a pulling force or tension from the proximal end of the cable 30 to the distal end 32 thereof.

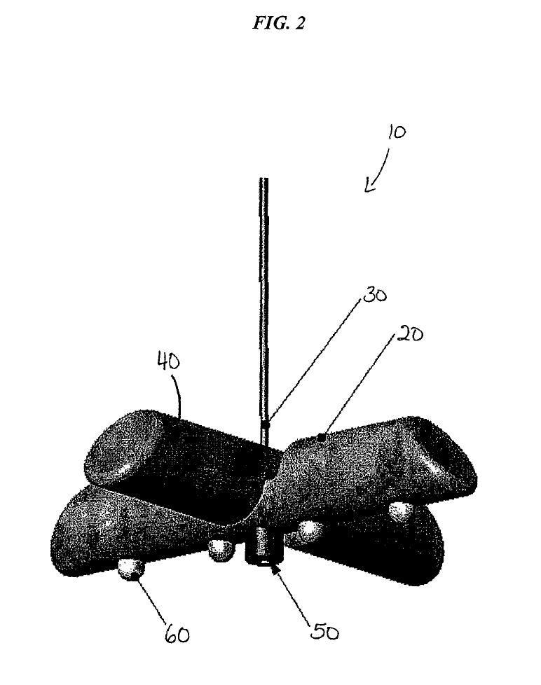

FIG. 2 illustrates an exemplary embodiment of the device 10 in a deployed configuration. In particular, the second elongate member 40 is shown in the second position, as previously discussed in respect to FIG. 1, in which the first elongate member 20 and the second elongate member 40 mate to one another and are configured to engage tissue. With further reference to FIG. 1, to enable mating of the elongate members, the first elongate member 20 can include a first surface having a first mating structure 26 formed therein and the second elongate member 40 can include a second surface having a second mating structure 46 formed therein. The second mating structure 46 can be complementary to the first mating structure 26. Further, the first and second mating structures 26, 46 can be configured to engage each other when the first and second elongate members 20, 40 are operatively mated. In general, the first and second mating structures, 26, 46 can be formed from a variety of configurations sufficient to interlock the first and second elongate members, 20, 40 in a desired orientation. FIG. 2 illustrates an exemplary embodiment in which the first and second elongate members 20, 40 are interlocked with one another by a dovetail joint. However, one skilled in the art will appreciate that the first and second elongate members can be mated to one another using a variety of known techniques.

As shown in FIG. 2, the first and second elongated members 20, 40 can be mated such that they are positioned relative to one another in a substantially perpendicular orientation. However, one skilled in the art will appreciate that the elongate members 20, 40 can be mated in a variety of orientations sufficient to enable the device 10 to engage and manipulate tissue.

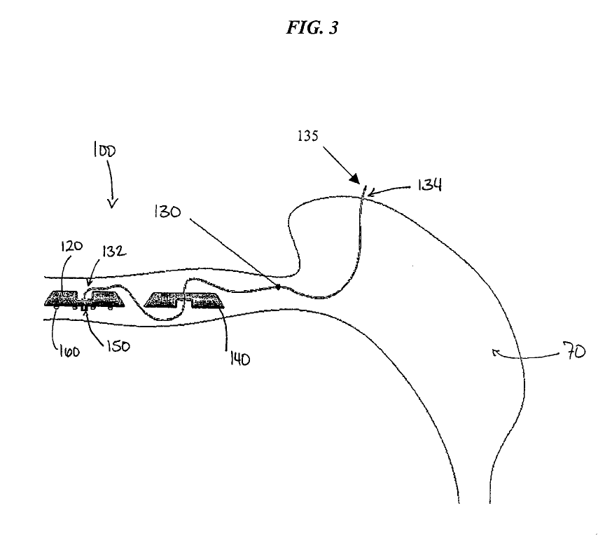

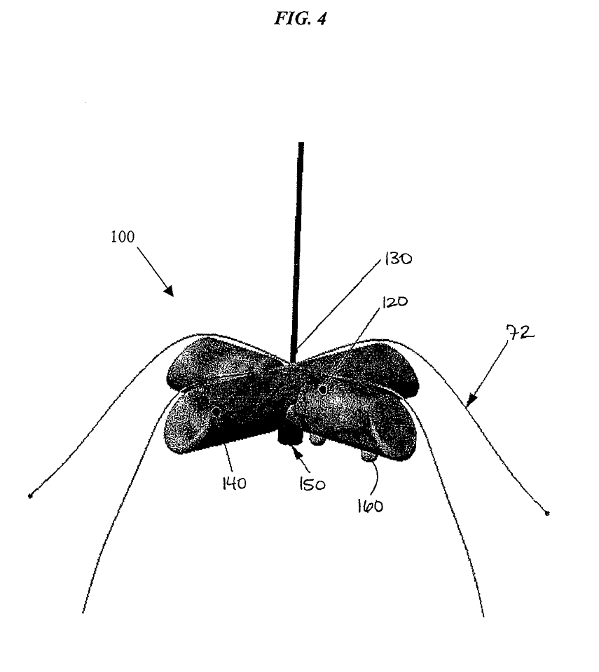

Exemplary methods for creating a space in a body cavity and tenting a wall of a body cavity are further described with reference to FIGS. 3 and 4. After preparing a patient for surgery, a tissue-tenting apparatus 100 can be delivered to a surgical site, such as the stomach 70, through a natural body orifice. As illustrated in FIG. 3, the tissue-tenting apparatus 100 can include a first elongate member 120 and a second elongate member 140 coupled to a cable 130. The tissue-tenting apparatus 100 can further include an imaging apparatus 150 and at least one illumination element 160 coupled thereto. One skilled in the art will appreciate that the tissue-tenting apparatus 100 can have any shape and configuration, as described above with respect to device 10. FIG. 3 further illustrates the tissue-tenting apparatus 100 in a delivery configuration in which the second elongate member 140 is slidably disposed on the cable 130 and positioned proximally to the first elongate member 120 which is positioned on a distal end 132 of the cable 130.

Following the delivery of the tissue-tenting apparatus 100 to the stomach 70, a proximal portion 134 of the cable 130, which can include a piercing tip 135, can be passed through the stomach region to pierce the stomach wall and the peritoneal wall, such that the proximal portion 134 of the cable 130 exits the stomach 70 and the body of the patient.

Upon transmural passage of the cable 130, tension, such as a pulling force, can be exerted on the proximal portion 134 thereby manipulating the cable 130 to configure the tissue-tenting apparatus 100 in a deployed configuration such that at least a portion of the tissue-tenting apparatus 100 is capable of lifting tissue at the surgical site. As illustrated in FIG. 4, the application of tension to the cable 130 results in the tissue-tenting apparatus 100 being drawn toward a surface wall 72 of the stomach 70 and coming into contact therewith, such that the second elongate member 140 is manipulated to mate with the first elongate member 120. Continued exertion of a pulling force on the cable 130 results in the deployed tissue-tenting apparatus 100 lifting the wall 72 of the stomach 70, thereby creating a space within the stomach 70.

Once the stomach wall 72 is tented as illustrated in FIG. 4, an imaging apparatus 150 can be activated to enable visualization within the stomach 70. In general, the imaging apparatus 150 provides a top-down view of the internal surgical site. An illumination source 160 associated with the tissue-tenting apparatus 100 can also be activated to provide illumination and visibility to the tented stomach 70.

The method described above can provide many advantages. For example, the creation of a space in a body cavity using a mechanical wall lift avoids the need for insufflation techniques when performing minimally invasive surgical procedures. In addition, the provision of illumination elements and an imaging apparatus on a wall lift provide enhanced illumination and visibility to an internal surgical site. While the above method has been described in terms of the manipulation and creation of space in the stomach, one skilled in the art will appreciate that the methods and devices described herein are equally applicable to the manipulation of other tissue or body cavities in a variety of minimally invasive surgical procedures.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A device for manipulating tissue, comprising:
a first elongate member with a cable coupled thereto;
a second elongate member slidably disposed on the cable and movable between a first position in which the first elongate member and second elongate member are configured to be inserted through a hollow tube, and a second position in which the first elongate member and second elongate member mate to one another and are configured to engage tissue and the cable can be manipulated to move the tissue; and
an imaging apparatus disposed on at least one of the first elongate member, the second elongate member, and the cable.

2. The device of claim 1, wherein the first elongate member and the second elongate member mate in a substantially perpendicular orientation.

3. The device of claim 1, wherein the imaging apparatus is at least one of a CCD chip and a CMOS chip.

4. The device of claim 1, wherein at least one of the first elongate member, the second elongate member, and the cable further comprises at least one illumination element coupled thereto.

5. The device of claim 4, wherein the at least one illumination element is a light-emitting diode.

6. The device of claim 1, wherein a proximal end of the cable comprises a piercing tip.

7. The device of claim 1, wherein a first surface of the first elongate member has a first mating structure formed therein and a mating surface of the second elongate member has a second mating structure formed thereon, the second mating structure being complementary with the first mating structure.

8. The device of claim 7, wherein the first and second mating structures are configured to engage each other when the first and second elongate members are operatively mated.

9. The device of claim 1, wherein the cable communicates a signal from the imaging apparatus.

10. The device of claim 1, wherein a wire communicating a signal from the imaging apparatus is adjacent the cable.

11. A device for tenting a wall of a body cavity, comprising:
a first elongate member having a cable coupled to an intermediate portion thereof; and
an imaging apparatus disposed on at least one of a portion of the first elongate member and the cable, the cable and the first elongate member being configured to cooperate to move tissue.

12. The device of claim 11, further comprising a second elongate member slidably disposed on the cable proximal to the first elongate member and configured to be manipulated to mate with the first elongate member upon the application of tension to the cable.

13. The device of claim 11, wherein the first elongate member and the second elongate member mate in a substantially perpendicular orientation.

14. The device of claim 11, wherein the imaging apparatus is at least one of a CCD chip and a CMOS chip.

15. The device of claim 11, wherein at least one of the first elongate member, the second elongate member, and the cable further comprises at least one illumination element coupled thereto.

16. The device of claim 11, wherein the at least one illumination element is a light-emitting diode.

17. A method for creating a space in a body cavity, comprising:
delivering a tissue tenting apparatus coupled to a cable in a delivery configuration to a surgical site through a natural body orifice;
manipulating the cable to configure the tissue tenting apparatus in a deployed configuration such that at least a portion of the tenting apparatus is capable of lifting tissue at the surgical site; and
activating an imaging apparatus formed on one of the tenting apparatus and the cable to enable visualization of the surgical site.

18. The method of claim 17, wherein the surgical site is the stomach.

19. The method of claim 17, wherein manipulating the cable is effected by breaching the stomach wall and the peritoneal wall with a proximal portion of the cable such that it exits the body cavity and exerting a pulling force on the cable.

20. The method of claim 17, wherein the tenting apparatus, in the deployed configuration, is in the form of a first and second elongate members mated to one another in a substantially perpendicular orientation.
